(19) United States
(12) Patent Application Publication
(10) Pub. No.: US 2009/0299468 A1
(43) Pub. Date: Dec. 3, 2009

(54) ENDOPROSTHESIS COATING

(75) Inventors: Scott R. Schewe, Eden Prairie, MN (US); Jan Weber, Maastricht (NL); Michele Zoromski, Minneapolis, MN (US); Robert W. Warner, Woodbury, MN (US)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 12/469,203

(22) Filed: May 20, 2009

Related U.S. Application Data
(60) Provisional application No. 61/056,912, filed on May 29, 2008.

(51) Int. Cl. A61F 2/06; B05D 1/24

(52) U.S. Cl. 623/1.46; 427/2.24

(57) ABSTRACT

An endoprosthesis, e.g., a stent (e.g., a drug eluting stent), includes a surface, a first layer having a polyelectrolyte deposited on the surface, a plurality of polymeric particles deposited on the first layer, and a coating of a porous material deposited on the plurality of polymeric particles. At least one particle of the plurality of polymeric particles includes a polymer matrix and a drug distributed in the polymer matrix. A method of making the endoprosthesis is also disclosed.

### CROSS-REFERENCE TO RELATED APPLICATIONS

This application claims priority under 35 USC §119(e) to U.S. Provisional Patent Application Ser. No. 61/056,912, filed on May 29, 2008, the entire contents of which are hereby incorporated by reference.

### TECHNICAL FIELD

This invention relates to coating endoprostheses.

### BACKGROUND

The body includes various passageways including blood vessels such as arteries, and other body lumens. These passageways sometimes become occluded or weakened. For example, they can be occluded by a tumor, restricted by plaque, or weakened by an aneurysm. When this occurs, the passageway can be reopened or reinforced, or even replaced, with a medical endoprosthesis. An endoprosthesis is an artificial implant that is typically placed in a passageway or lumen in the body. Many endoprostheses are tubular members, examples of which include stents, stent-grafts, and covered stents.

Many endoprostheses can be delivered inside the body by a catheter. Typically the catheter supports a reduced-size or compacted form of the endoprosthesis as it is transported to a desired site in the body, for example the site of weakening or occlusion in a body lumen. Upon reaching the desired site, the endoprosthesis is installed so that it can contact the walls of the lumen. Stent delivery is further discussed in Heath, U.S. Pat. No. 6,290,721, the entire contents of which is incorporated herein by reference.

One method of installation involves expanding the endoprosthesis. The expansion mechanism used to install the endoprosthesis may include forcing it to expand radially. For example, the expansion can be achieved with a catheter that carries a balloon in conjunction with a balloon-expandable endoprosthesis reduced in size relative to its final form in the body. The balloon is inflated to deform and/or expand the endoprosthesis in order to fix it at a predetermined position in contact with the lumen wall. The balloon can then be deflated, and the catheter withdrawn.

It is sometimes desirable for an endoprosthesis to contain a therapeutic agent, or drug which can elute into the body fluid in a predetermined manner once the endoprosthesis is implanted.

### SUMMARY

In one aspect, the invention features an endoprosthesis, e.g., a stent (e.g., a drug eluting stent), that includes a surface, a first layer having a polyelectrolyte deposited on the surface, a plurality of polymeric particles deposited on the first layer, and a coating of a porous material deposited on the plurality of polymeric particles. At least one particle of the plurality of polymeric particles includes a polymer matrix and a drug distributed in the polymer matrix.

In another aspect, the invention features a method of making an endoprosthesis. The method includes applying a first layer of a polyelectrolyte to a surface; applying a plurality of bioerodible particles to the first layer; and forming a porous top coating over the particles.

Embodiments may include one or more of the following features. The endoprosthesis further comprises a second layer comprising a polyelectrolyte deposited between the coating and the plurality of polymeric particles. The porous material of the coating is formed by an in situ sol-gel process in the second layer. The second layer further comprises the porous material. The first layer further comprises the porous material. The polymer matrix is formed of a bioerodible polymer. The porous material comprises oxides and hydroxides of titanium, iridium, zirconium, ruthenium, hafnium, silicon, and aluminum. The surface is formed of a metal. The metal is stainless steel, nitinol, tungsten, tantalum, rhenium, iridium, silver, gold, bismuth, platinum or alloys thereof. At least one particle has a diameter of about 10 nm to about 1 μm. The plurality of polymeric particles form a layer with a thickness of about 10 nm to about 1 μm. The first layer has a thickness of about 1 nm to about 100 nm. The porous material has a porosity of about 90% or less. The porous material has a porosity of about 30% or more.

Embodiments may include one or more of the following features. The method further comprises applying a second layer of a polyelectrolyte to the particles and applying a precursor composition over the second layer to form the porous top coating via an in situ sol-gel reaction. At least one of the bioerodible particles comprises a polymeric matrix and a drug dispersed in the matrix. The method further comprises forming the at least one of the bioerodible particles with the drug dispersed in the matrix by emulsion-polymerization. The method comprises forming the porous top coating by applying a sol-gel precursor composition to form a wet gel. The method further comprises converting the wet gel to a ceramic or ceramic-like material by drying the wet gel. The precursor composition comprises a metal oxide precursor. The metal oxide precursor is tetraethylorthosilicate, titanium isopropoxide, or iridium acetylacetonate. The method comprises applying the first layer and the particles by LBL deposition. The method comprises applying the second layer by LBL deposition. The particle has a size of about 10 nm to about 1 μm. The first layer has a thickness of about 1 nm to about 100 nm. The porous top coating has a porosity of about 90% or less. The porous top coating has a porosity of about 30% or more.

Aspects and/or embodiments may have one or more of the following additional advantages. The endoprosthesis, e.g., a drug eluting stent, can include a plurality of drug-incorporating particles covered by a porous top coating to facilitate drug elution. The drug can be incorporated into the particles before the particles are applied to the stent, such that the more onerous drug-loading after the formation of stent coatings can be avoided. The particles can be formed of bioerodible materials such as bioerodible polymers. The top coating can be formed of a ceramic, e.g. IROX and TiOx, which can have therapeutic advantages such as reducing the likelihood of restenosis and enhancing endothelialization. The drug elution profile over time can be selected by controlling the porosity and/or thickness of the top coating as well as the size of the particles and/or drug distribution in the particles. For example, top coatings having the same porosity but greater thickness will release drugs at a relatively slower rate than coatings with relatively smaller thickness; while drug-incorporating particles clad with thicker bioerodible polymer will release drugs at a later stage than the particles clad with thinner bioerodible polymer. The top coating can have multiple layers of porous materials with different porosities to further control drug release. Multiple layers of drug-incorporating bioerodible particles with multiple types of drugs can be applied to the stent surface to obtain selected therapeutic effect. The endoprostheses may be fully endothelialized or may be bioerodible, thus not needing to be removed from a lumen after implantation.

Other aspects, features, and advantages will be apparent from the description and drawings, and from the claims.

### DESCRIPTION OF DRAWINGS

FIGS. 1A-1C are longitudinal cross-sectional views, illustrating delivery of a stent in a collapsed state, expansion of the stent, and the deployment of the stent in a body lumen.

FIG. 2 is a perspective view of an embodiment of a stent.

FIG. 3 is a schematic cross-sectional view of the surface of the stent.

FIG. 4 is a flow chart of an embodiment of a method of making a stent.

Like reference symbols in the various drawings indicate like elements.

### DETAILED DESCRIPTION

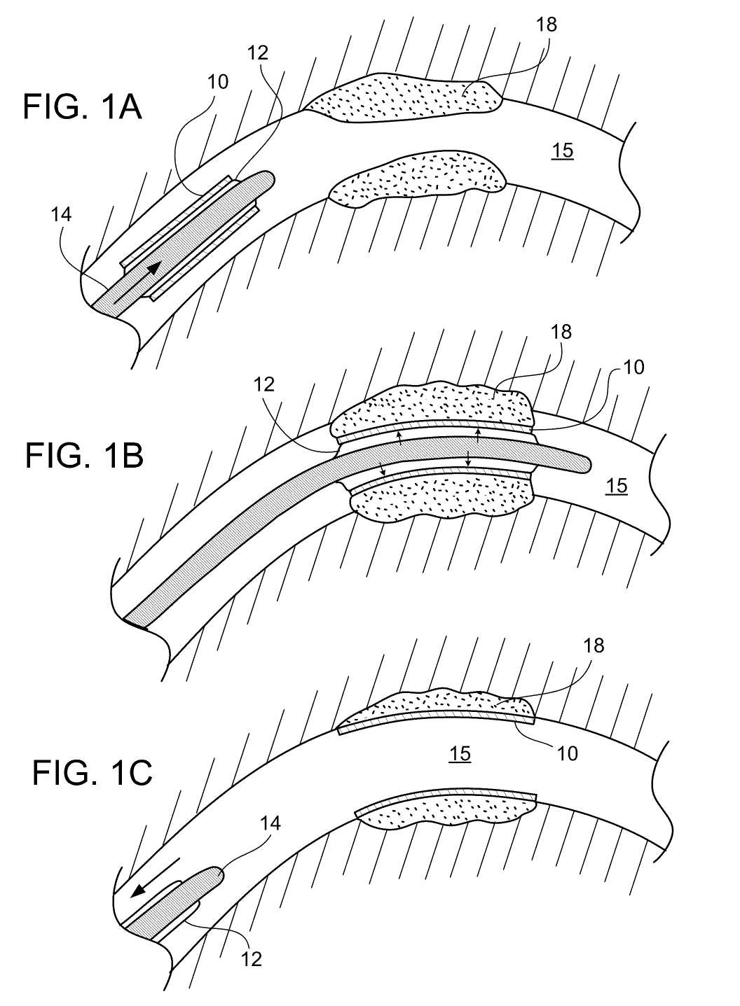

Referring to FIGS. 1A-1C, a stent 10 is placed over a balloon 12 carried near a distal end of a catheter 14, and is directed through the lumen 15 (FIG. 1A) until the portion carrying the balloon and stent reaches the region of an occlusion 18. The stent 10 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

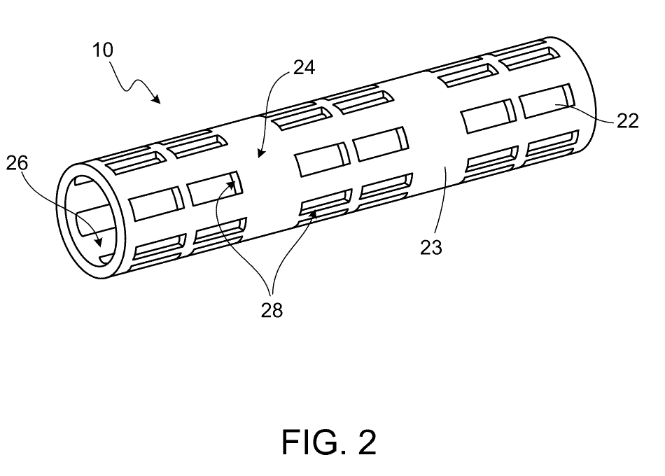

Referring to FIG. 2, the stent 10 includes a plurality of fenestrations 22 defined in a wall 23. Stent 10 includes several surface regions, including an outer, or abluminal, surface 24, an inner, luminal, surface 26, and a plurality of cutface surfaces 28. The stent can be balloon expandable, as illustrated above, or a self-expanding stent. Examples of stents are described in Heath '721, supra.

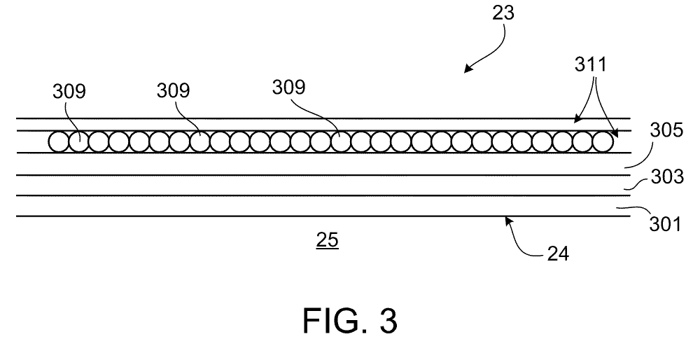

Referring to FIG. 3, a cross-sectional view, in a particular embodiment, a stent wall 23 includes a stent body 25 formed, e.g. of a metal, and includes an undercoating of multiple layers of polyelectrolytes 301, 303, and 305 formed on a surface of the stent, e.g., the abluminal surface 24, e.g. by the method described in more detail below. On top of the polyelectrolyte layers are a plurality of particles 309 formed, e.g., of a bioerodible polymer, covered by a top coating 311 of a porous material.

In embodiments, the particles 309 can be substantially spherical and each individual particle can have a diameter of at least about 5 nanometer (“nm”), e.g., at least about 10 nm, at least about 100 nm, at least about 1 micrometer (“micron” or “μm”), or at least about 5 μm, and/or at most about 10 μm (e.g., at most about 5 μm, at most about 1 μm, at most about 500 nm, at most about 100 nm, at most about 10 nm). The particles 309 can include a drug dispersed in the bioerodible polymer matrix. The drug distribution in the polymer particle can be selected to be, e.g., uniform or non-uniform, such that the drug can be released from the polymer particles in a predetermined fashion. For example, to have a steady increase of drug release rate, the drug can have a higher concentration in the interior region of the particle (e.g., a region from the center to about two thirds of the particle radius) than at the surface region (e.g., a region from about two thirds of the particle radius to the particle surface) of the particle, e.g., about 60%-100% of the drug in the interior region with about 40%-0% of the drug at the surface region. The drug release profile can also be controlled by varying the diameters of the particles. In embodiments, the diameters of the particles can be uniform or non-uniform. For example, particles with larger diameters that erode over longer periods of time can enable a more extended drug release profile. In some embodiments, one or more drugs can be incorporated into an individual particle. In other embodiments, multiple particles are used where each individual particle contains one drug that can be different from that contained in other particles. In embodiments, the particles can be arranged in a single layer or in multiple layers. For example, multiple layers of particles containing different drugs in different layers can be used to obtain a specific predetermined therapeutic effect. For another example, a multi-layer configuration can be formed by alternating a layer of polyelectrolyte and a layer of drug-incorporating polymer particles. The particle layer or layers can have a thickness of at least about 5 nm (e.g., at least about 10 nm, at least about 100 nm, at least about 500 nm, or at least about 900 nm) and/or at most about 1 micrometer (e.g., at most about 750 nm, at most about 500 nm, at most about 100 nm, at most about 50 nm, at most about 25 nm). The thickness can be uniform or non-uniform. For example, the thickness can increase from one end of the endoprosthesis to another end in an overall linear manner, an overall non-linear manner (e.g., an overall parabolic increase, an overall exponential increase), or a stepwise manner. Additionally or alternatively, the particles can have other shapes, such as shapes of a rod, a cube, an oval, or a coil with at least one dimension defined in the nanometer scale (e.g., about 1 to about 1000 nm). The particles can also be charged.

Examples of bioerodible polymers include polylactic acid (PLA), polyglycolic acid (PGA), polylactic glycolic acid (PLGA), polycaprolactone (PCL), polyorthoesters, polydioxanone, poly(trimethylene carbonate) (PTMC), polyphosphazenes, polyketals, proteins (e.g., fibrin, collagen, gelatin), polysaccharides (e.g., chitosan and hyaluronan), polyanhydrides (e.g., poly(ester anhydride)s, fatty acid-based polyanhydrides, amino acid-based polyanhydrides), polyesters, polyester-polyanhydride blends, polycarbonate-polyanhydride blends, and/or combinations thereof. Other drug-incorporating bioerodible particles include liposomes (i.e., phospholipid vesicles) and solid lipid nanoparticles (SLNs).

The undercoating of multiple layers of polyelectrolytes, e.g., layers 301, 303, and 305, are applied by a layer-by-layer (“LbL”) process which is discussed in detail below. The number of layers of polyelectrolytes, and therefore the layer thickness, can be selected in order to obtain suitable charge density for allowing subsequent deposition of the polymer particles, e.g., charged particles. The thickness of the polyelectrolyte layers can be controlled to at least about 1 nm (e.g., at least about 5 nm, at least about 10 nm, at least about 100 nm, at least about 500 nm, or at least about 900 nm) and/or at most about 1 micrometer (e.g., at most about 750 nm, at most about 500 nm, at most about 100 nm, at most about 50 nm, at most about 25 nm). The thickness can be uniform or non-uniform. For example, the thickness can increase from one end of the endoprosthesis to another end in an overall linear manner, an overall non-linear manner (e.g., an overall parabolic increase, an overall exponential increase), or a stepwise manner. In some embodiments, the polyelectrolyte layers partially cover the endoprosthesis body. For example, the layer can cover at least about 10 percent (e.g., at least about 20 percent, at least about 30 percent, at least about 40 percent, at least about 50 percent, at least about 60 percent, at least about 70 percent, at least about 80 percent, at least about 90 percent, or at least about 95 percent) and/or at most 100 percent (e.g., at most about 95 percent, at most about 90 percent, at most about 80 percent, at most about 70 percent, at most about 60 percent, at most about 50 percent, at most about 40 percent, at most about 30 percent, or at most 20 percent) of the surface (e.g., abluminal surface) area of the endoprosthesis body. In embodiments, an additional over-coating of multiple layers of polyelectrolytes can be deposited over the drug-incorporating polymer particles, e.g., by an LbL process.

The top coating 311 can be a porous material which is formed by an in situ sol-gel reaction, e.g., in the over-coating polyelectrolyte layers, and/or in the undercoating polyelectrolyte layers. In embodiments, the thickness of the top coating 311 is in the range of about 1 nm to 500 nm, e.g., about 10 nm to 50 nm. The porous material can have an open-cell porous structure, so that the pores are interconnected and serve as passageways for body fluid to reach the drug-incorporating particles 309 underneath. The average diameter of pores can vary from about 0.5 nm to 500 nm, e.g., 1 nm to 100 nm. In embodiments, the ratio of the volume of pores to the total volume of the surface region, or porosity, of the top coating 311 is about 90% or less (e.g., about 80%, about 70%, about 60%, about 50% or less). The porous material can be an inorganic material in the form of a sol or a gel. In embodiments, the porous material is IROX, which can have therapeutic benefits such as enhancing endothelialization. IROX and other ceramics are discussed further in Alt, et al., U.S. Pat. No. 5,980,566.

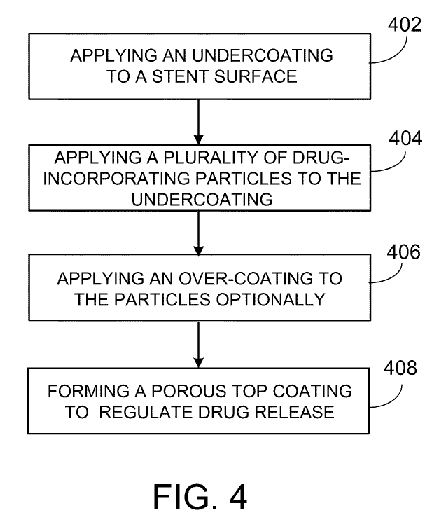

Referring to FIG. 4, the stent is formed by first applying an undercoating of one or more polyelectrolyte layers to a surface of the stent (step 402), followed by applying a plurality of drug-incorporating particles, e.g., charged bioerodible polymer nanoparticles (step 404). Next, an over-coating of one or more polyelectrolyte layers can be optionally applied to the polymer particles (step 406), and a porous top coating is formed, e.g., by applying a precursor composition for an in situ sol-gel reaction to the over-coating (step 408).

In steps 402, 404, and 406, charged layers containing polyelectrolytes sandwiching the drug-incorporating polymer particles are assembled upon the stent surface, e.g., the abluminal surface, using a layer-by-layer (“LBL”) technique in which the layers electrostatically self-assemble. In the LBL technique, a first layer having a first net surface charge is deposited on an underlying substrate, followed by a second layer having a second net surface charge that is opposite in sign to the net surface charge of the first layer. Thus, the charge on the outer layer is reversed upon deposition of each sequential layer. Additional first and second layers can then be alternatingly deposited on the substrate to build multi-layered structure to a predetermined or targeted thickness. For example, in steps 402 and 406, deposited undercoating and over-coating can either be a polyelectrolyte monolayer or polyelectrolyte multilayers (“PEM”) with thickness ranging from 0.3 nm to 1 μm, e.g., 1 nm to 500 nm. In step 404, charged drug-incorporating polymer particles, e.g., bioerodible polymer nanoparticles, with net surface charges opposite in sign to those of the undercoating and over-coating, are deposited.

In certain embodiments, the LBL assembly can be conducted by exposing a selected charged substrate (e.g., stent) to solutions or suspensions that contain species of alternating net charge, including solutions or suspensions that contain charged templates (e.g., polystyrene spheres), polyelectrolytes, and, optionally, charged therapeutic agents. The concentration of the charged species within these solutions and suspensions, which can be dependent on the types of species being deposited, can range, for example, from about 0.01 mg/mL to about 100 mg/mL (or to about 50 mg/mL, or to about 30 mg/mL). The pH of these suspensions and solutions can be such that the templates, polyelectrolytes, and optional therapeutic agents maintain their charge. Buffer systems can be used to maintain charge. The solutions and suspensions containing the charged species (e.g., solutions/suspensions of templates, polyelectrolytes, or other optional charged species such as charged therapeutic agents) can be applied to the charged substrate surface using a variety of techniques. Examples of techniques include spraying techniques, dipping techniques, roll and brush coating techniques, techniques involving coating via mechanical suspension such as air suspension, ink jet techniques, spin coating techniques, web coating techniques and combinations of these processes. Layers can be applied over an underlying substrate by immersing the entire substrate (e.g., stent) into a solution or suspension containing the charged species, or by immersing half of the substrate into the solution or suspension, flipping the same, and immersing the other half of the substrate into the solution or suspension to complete the coating. In some embodiments, the substrate is rinsed after application of each charged species layer, for example, using a washing solution with a pH that maintains the charge of the outer layer.

Polyelectrolytes are polymers having charged (e.g., ionically dissociable) groups. The number of these groups in the polyelectrolytes can be so large that the polymers are soluble in polar solvents (including water) when in ionically dissociated form (also called polyions). Depending on the type of dissociable groups, polyelectrolytes can be classified as polyacids and polybases. When dissociated, polyacids form polyanions, with protons being split off. Polyacids include inorganic, organic and biopolymers. Examples of polyacids are polyphosphoric acids, polyvinylsulfuric acids, polyvinylsulfonic acids, polyvinylphosphonic acids and polyacrylic acids. Examples of the corresponding salts, which are called polysalts, are polyphosphates, polyvinylsulfates, polyvinylsulfonates, polyvinylphosphonates and polyacrylates. Polybases contain groups that are capable of accepting protons, e.g., by reaction with acids, with a salt being formed. Examples of polybases having dissociable groups within their backbone and/or side groups are polyallylamine, polyethylimine, polyvinylamine and polyvinylpyridine. By accepting protons, polybases form polycations. Some polyelectrolytes have both anionic and cationic groups, but nonetheless have a net positive or negative charge.

The polyelectrolytes can include those based on biopolymers. Examples include alginic acid, gum arabicum, nucleic acids, pectins and proteins, chemically modified biopolymers such as carboxymethyl cellulose and lignin sulfonates, and synthetic polymers such as polymethacrylic acid, polyvinylsulfonic acid, polyvinylphosphonic acid and polyethylenimine. Linear or branched polyelectrolytes can be used. Using branched polyelectrolytes can lead to less compact polyelectrolyte multilayers having a higher degree of wall porosity. In some embodiments, polyelectrolyte molecules can be crosslinked within or/and between the individual layers, to enhance stability, e.g., by crosslinking amino groups with aldehydes. Furthermore, amphiphilic polyelectrolytes, e.g., amphiphilic block or random copolymers having partial polyelectrolyte character, can be used in some embodiments to affect permeability towards polar small molecules.

Other examples of polyelectrolytes include low-molecular weight polyelectrolytes (e.g., polyelectrolytes having molecular weights of a few hundred Daltons up to macromolecular polyelectrolytes (e.g., polyelectrolytes of synthetic or biological origin, which commonly have molecular weights of several million Daltons). Still other examples of polyelectrolyte cations (polycations) include protamine sulfate polycations, poly(allylamine)polycations (e.g., poly(allylamine hydrochloride) (PAH)), polydiallyldimethylammonium polycations, polyethyleneimine polycations, chitosan polycations, gelatin polycations, spermidine polycations and albumin polycations. Examples of polyelectrolyte anions (polyanions) include poly(styrenesulfonate) polyanions (e.g., poly(sodium styrene sulfonate) (PSS)), polyacrylic acid polyanions, sodium alginate polyanions, eudragit polyanions, gelatin polyanions, hyaluronic acid polyanions, carrageenan polyanions, chondroitin sulfate polyanions, and carboxymethylcellulose polyanions.

Still more examples of polyelectrolytes and LBL assembly are disclosed in commonly assigned U.S. Publication No. 2006/0100696 A1, the entire disclosure of which is incorporated herein by reference.

The drug-incorporating polymer particles (e.g., particles 309 in FIG. 3) can be obtained by various polymerization techniques, such as dispersion-, suspension-, or emulsion-polymerization. For example, polymer nanoparticles and/or microparticles formed of a bioerodible polymer, such as PLGA, encapsulating a drug, such as paclitaxel, can be produced via a classical emulsion solvent-evaporation/extraction method. In such a method, an emulsifier such as an amphiphilic surfactant (e.g., possessing both hydrophilic and hydrophobic groups in the same molecule) is usually used to stabilize the nanoparticles and/or microparticles formed in the emulsification process, e.g., the separation process of two phases (oil/water) to form the emulsion or particles. The emulsifier can inhibit coalescence of droplets, and control the particle size, the morphological properties of the particles, and drug-release properties of the particles. Examples of emulsifier include poly(vinyl alcohol) (PVA), D-α-tocopheryl polyethylene glycol 1000 succinate (vitamin E TPGS), and Poloxamer 188 (Sympersonic® F68). More examples of producing the drug-incorporating polymer particles are disclosed, e.g., in Fonseca, et al., Journal of Controlled Release, 83: 273-286 (2002); Mu, et al., Journal of Controlled Release, 80: 129-144 (2002); Mu, et al., Journal of Controlled Release, 86: 33-48 (2003); and Freitas, International Journal of Pharmaceutics, 295: 201-211 (2005). Other suitable bioerodible matrix materials for drug delivery include liposomes and solid lipid nanoparticles (SLN) as mentioned above. SLN are particles made from solid lipids with a diameter between about 50 and 1000 nm. A detailed description of SLN is provided, e.g., in Muller, et al., European Journal of Pharmaceutics and Biopharmaceutics, 50: 161-177 (2000).

The drug-incorporating particles can be charged as produced, or charges can be introduced, e.g., by conjugating polymer particles with a charged molecule or by coating the particles with polyelectrolytes. As a result, the particles can be applied to the undercoating via the LbL process, as in step 404. In other embodiments, referring back to FIG. 3, the drug-incorporating particles 309 can be electrically neutral and can be applied to the undercoating (e.g., one or more of the layers 301, 303, and 305) via absorption. For example, a hydrophobic or ultrahydrophobic undercoating can be applied on the stent surface and be used to absorb or attach drug-incorporating SLNs, which are naturally hydrophobic, to the surface. There are many techniques to generate a surface that is hydrophobic or ultrahydrophobic. One exemplary method is the use of layer-by-layer techniques. Select surface roughness is required to generate hydrophobicity and it may be created in the LbL techniques by depositing one or more layers of particles. A variety of particles are available for this purpose, including, for example, carbon, ceramic and metallic particles, which may be in the form of plates, cylinders, tubes, and spheres, among other shapes. In some embodiments, charged particle layers are introduced as part of the layer-by-layer process. Certain particles, such as clays, have an inherent surface charge. Surface charge may also be provided, if desired, by attaching species that have a net positive or negative charge to the particles, e.g., by adsorption, covalent bonding, and so forth.

One specific layer-by-layer technique for forming superhydrophobic surfaces on underlying substrates is described in R. M. Jisr, et al., “Hydrophobic and Ultrahydrophobic Multilayer Thin Films from Perfluorinated Polyelectrolytes,” Angew. Chem. Int. Ed. 2005, 44, 782-785. The polyelectrolytes employed are poly(diallyidimethylammonium) (PDADMA), and a polycation synthesized from poly(vinylpyridine) and a fluorinated alkyl iodide. Attapulgite, a negatively charged clay mineral with a needlelike morphology, is used to form the particulate layers. As is typical of layer-by-layer processes, polyelectrolytes are deposited under ambient conditions using dilute solutions/dispersions, in this case in methanol. Three groups of bilayer pairs are deposited. The first, adjacent to the substrate, consists of several PDADMA/PSS bilayers. This is followed by additional bilayers of clay particles and PDADMA, which produces surface roughness, and is in turn followed by bilayers of fluorinated polyelectrolytes, specifically the nafion and PFPVP. No annealing steps are required. The resulting surface has advancing and receding water contact angles in excess of 140°, even after 2 months of immersion in water. More examples of creating hydrophobic and super hydrophobic surface are disclosed in Weber, et al., U.S. Publication No. 2007/0005024 A1.

Referring particularly to step 408 of FIG. 4, in embodiments, a porous top coating can be formed by applying a precursor composition for an in situ sol-gel reaction to the stent surface with deposited polyelectrolytes layers and drug-incorporating particles. The precursor composition permeates the polyelectrolytes layers and an in situ sol-gel reaction can take place within the interstices of polyelectrolytes to form a porous material which can be an inorganic material (e.g., oxides, nitrides, or hydroxides) in the form of a sol or a gel. In other embodiments, when an over-coating is not applied (i.e., step 406 is omitted), a porous top coating can still be formed by a sol-gel process. The sol-gel process is a versatile solution process for making ceramic and glass materials. In general, the sol-gel process involves the transition of a system from a liquid “sol” (mostly colloidal) into a solid “gel” phase. The starting materials or precursors used in the preparation of the sol are usually inorganic metal salts or metal organic compounds such as metal alkoxides. In a typical sol-gel process, the precursor is subjected to a series of hydrolysis and/or polymerization reactions to form a colloidal suspension, or a sol. Further processing of the sol results in ceramic materials in different forms. For example, thin films can be produced on a substrate (e.g., a stent or pre-stent material, such as metal tube) by spin coating, roll coating, inkjet printing or spraying with the sol. To obtain selective coating, e.g., coating of the abluminal surface only, instead of using dip coating within a solution, sol can be printed on the desired surface of the stent. When the colloidal particles in sol condense in a new phase, a wet gel will form, with a solid macromolecule immersed in a solvent. With further drying and heat-treatment, the gel is converted into a dense ceramic film. In embodiments, to diminish the likelihood of heat damage to drug incorporated in the polymer particles, either porous material in the form of sol or wet gel may be used as the top coat over the particles or a low-temperature drying process, e.g., vacuum solvent extraction, is applied to form a ceramic or ceramic-like material. In general, sol-gel-derived ceramic porous layers are generated with use of an organic template or a surfactant used as a template which needs to be removed at high temperatures, such as polyethylene glycol (PEG), polyvinylpyrrolidone (PVP), polyelectrolyte materials and oil emulsions. In embodiments, other thin film processes may be employed in formation of the layers that modulate drug release and enhance healing. Examples include: CVD (chemical vapor deposition), PVD (physical vapor deposition), and other thin film processes that provide a thin porous film similar to that obtained through the sol-gel process.

An exemplary precursor composition is a mixture of ethanol, a precursor (e.g., tetraethylorthosilicate or TEOS, titanium isopropoxide, iridium acetylacetonate, etc.) with the molar ratio of ethanol to precursor ranging from about 1 to 16, and sometimes a small amount of water. A sol-gel reaction occurs when the precursor comes into contact with water molecules that hydrate or get trapped in the interstices of polyelectrolytes and decompose to form a gel or a ceramic deposit within the polyelectrolyte multilayers (PEM). Alternatively or additionally, the high charge density of polyelectrolytes attracts water molecules out of the precursor solution, and as a result, the local water content increases to such an extent that a sol-gel reaction or hydrolysis takes place and produces a gel or ceramic deposit around the polyelectrolytes, i.e., within the PEMs. For example, when an in situ reaction of a sol-gel solution of TEOS in pure ethanol with 15 wt. % water is carried out, the polyelectrolyte layers attract water because of their ionic charge, and their water content increases above 15 wt. %, thus activating the sol-gel reaction. This method is very controlled and stops automatically once the layers are saturated and charge density decreases.

Another exemplary precursor for use in the process can be titanium-based, e.g., titanium (IV) bis(ammonium lactate)dihydroxide (TALH), or titanium alkoxide such as titanium (IV) butoxide (Ti(OBu)₄), or titanium tetraisopropoxide (TTIP). In certain embodiments, as discussed above, if high-temperature processing is undesirable (e.g., if a stent already has a coating of heat sensitive elements, such as certain polymers, or drugs), water vapor treatment at relatively low temperature, e.g., about 60 to 180° C., is used to generate the ceramic layer. To improve the crystallinity and mechanical properties with the exposure to water vapor, silica sol can be introduced into the TiOₓ sol to form a porous TiOₓ-silica layer, as described in Imai, et al., J. Am. Ceram. Soc. 82:2301-2304, 1999. In yet other embodiments, to avoid the use of high temperatures, a non-polyelectrolyte compound, such as nonsurfactants, e.g., glucose, fructose, or urea, can be used to generate the porous ceramic coatings, as disclosed in Zheng, et al., J. Sol-Gel Science and Tech. 24:81-88, 2002. Glucose or urea can be removed with use of water at room temperature, to leave behind a pure porous ceramic coating. Similar to polyelectrolytes, selection of the non-polyelectrolyte compound can generate materials with different pore sizes, thus allowing generation of a desired drug release profile. For example, urea leaves relatively larger pores than glucose. Many nonsurfactants are biocompatible, and can also be allowed to remain in the sol-gel layer until they bioerode in the body after delivery of the stent.

Moreover, if the porous material is formed of ceramic titanium oxide (“TiOₓ”), the hydrophilicity or hydrophobicity of the layer can be selected accordingly to facilitate drug loading. Stents coated with TiOₓ and methods of coating stents with TiOₓ are described in the U.S. Patent Application No. 60/818,101, filed Jun. 29, 2006. As described therein, coating a stent with various combinations of hydrophobic and/or hydrophilic TiOₓ allows for placing various biologically active substances on selected regions of the stent. Following application of the TiOₓ coating, the medical device, e.g., a stent, can be exposed to conditions (e.g., UV light illumination) sufficient to cause desired regions of the device bearing TiOₓ coating to become hydrophilic or hydrophobic. In embodiments, the porous material can include, besides TiOₓ, e.g., TiO₂, other oxides, such as iridium oxide (IROX) and silica; or a combination of TiOₓ and IROX; or a combination of TiOx and ruthenium oxide (RuOₓ); or a combination of TiOₓ, IROX and RuOₓ. In embodiments, multiple layers of porous inorganic material with different porosity can be used as the top coating to control the drug release profile. Examples of sol-gel process are also provided, e.g., in Manoharan, et al., Proceedings of SPIE 3937: 44-50, 2000 and Guo, et al., Surface & Coating Technology 198:24-29, 2005. Other low-temperature techniques, such as pulsed laser deposition, can also be used to generate a porous top coating formed of ceramic or ceramic-like materials.

The ceramic or ceramic-like materials, such as iridium oxide (“IROX”), titanium oxide (“TiOₓ”), silicon oxide (“silica”) or oxides of niobium (“Nb”), tantalum (“Ta”), ruthenium (“Ru”) or mixture thereof. Certain ceramics, e.g. oxides, can reduce restenosis through the catalytic reduction of hydrogen peroxide and other precursors to smooth muscle cell proliferation. The oxides can also encourage endothelial growth to enhance endothelialization of the stent. When a stent is introduced into a biological environment (e.g., in vivo), one of the initial responses of the human body to the implantation of a stent, particularly into the blood vessels, is the activation of leukocytes, white blood cells which are one of the constituent elements of the circulating blood system. This activation causes an increase of reactive oxygen compound production. One of the species released in this process is hydrogen peroxide, H₂O₂, which is released by neutrophil granulocytes, which constitute one of the many types of leukocytes. The presence of H₂O₂ may increase proliferation of smooth muscle cells and compromise endothelial cell function, stimulating the expression of surface binding proteins which enhance the attachment of more inflammatory cells. A ceramic, such as IROX, can catalytically reduce H₂O₂. The morphology of the ceramic can enhance the catalytic effect and reduce proliferation of smooth muscle cells. In a particular embodiment, IROX is selected to form the coating 32, which can have therapeutic benefits such as enhancing endothelialization, while TiOₓ is selected to form coating 36, which can have a desirable porous structure to accommodate large volumes of drug. TiOₓ coatings are known to be blood-compatible, as disclosed in Matz, et al., Boston Scientific Corporation internal report, 2001; Tsyganov, et al., Surf. Coat. Tech. 200:1041-44, 2005. Blood compatible substances show only minor induction of blood clot formation. Titanium oxide-based surfaces may also promote endothelial cell adhesion, which, in turn, may reduce thrombogenicity of stents delivered to blood vessels, as disclosed in Chen, et al., Surf. Coat. Tech. 186:270-76, 2004. IROX and other ceramics are discussed further in Alt, et al., U.S. Pat. No. 5,980,566 and U.S. application Ser. No. 10/651,562, filed Aug. 29, 2003.

In some embodiment, the drug-incorporating particles are formed of non-bioerodible polymers and therefore the polymers can be removed following the sol-gel step while the drug remains covered by the porous top coating. A low-temperature removing process can be utilized, such as room-temperature wet etching or UV light exposure. In some embodiments, removing non-bioerodible polymers may involve long-term exposure of the polymers to an agitated aqueous solution with high salt concentrations at room temperature.

The terms “therapeutic agent,” “pharmaceutically active agent,” “pharmaceutically active material,” “pharmaceutically active ingredient,” “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), immunosuppressants (e.g., everolimus, tacrolimus, or sirolimus), anti-proliferative agents (e.g., cisplatin), and antibiotics (e.g., erythromycin). Additional examples of therapeutic agents are described in U.S. Publication No. 2005/0216074 A1. Polymers for drug elution coatings are also disclosed in U.S. Publication No. 2005/019265 A1.

The stents described herein can be configured for vascular, e.g. coronary and peripheral vasculature or non-vascular lumens. For example, they can be configured for use in the esophagus or the prostate. Other lumens include biliary lumens, hepatic lumens, pancreatic lumens, and urethral lumens.

Any stent described herein can be dyed or rendered radio-opaque by addition of, e.g., radio-opaque materials such as barium sulfate, platinum or gold, or by coating with a radio-opaque material. In embodiments, the porous structure can be formed directly on the stent body, as described above, or the porous structure can be formed in a coating over the stent body. The coating may be, e.g., a radio-opaque metal. The stent can include (e.g., be manufactured from) metallic materials, such as stainless steel (e.g., 316L, BioDur® 108 (UNS S29108), and 304L stainless steel, and an alloy including stainless steel and 5-60% by weight of one or more radioopaque elements (e.g., Pt, Ir, Au, W) (PERSS®) as described in U.S. Publication No. 2003/0018380 A1, U.S. Publication No. 2002/0144757 A1, and U.S. 2003/0077200 A1), Nitinol (a nickel-titanium alloy), cobalt alloys such as Elgiloy, L605 alloys, MP35N, titanium, titanium alloys (e.g., Ti-6Al-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003 (U.S. Publication No. 2005/0070990); and U.S. application Ser. No. 11/035,316, filed Jan. 3, 2005 (U.S. Publication No. 2006/0153729). Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003 (U.S. Publication No. 2004/0143317).

The stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., U.S. Pat. No. 6,290,721).

All publications, patent applications, patents, and other references mentioned herein are incorporated by reference herein in their entirety.

Still further embodiments are in the following claims.

What is claimed is:

1. An endoprosthesis, comprising:
a surface,
a first layer comprising a polyelectrolyte deposited on the surface,
a plurality of polymeric particles deposited on the first layer, and
a coating of a porous material deposited on the plurality of polymeric particles,
wherein at least one particle of the plurality of polymeric particles comprises a polymer matrix and a drug distributed in the polymer matrix.

2. The endoprosthesis of claim 1, further comprising a second layer comprising a polyelectrolyte deposited between the coating and the plurality of polymeric particles.

3. The endoprosthesis of claim 2, wherein the porous material of the coating is formed by an in situ sol-gel process in the second layer.

4. The endoprosthesis of claim 2, wherein the second layer further comprises the porous material.

5. The endoprosthesis of claim 1, wherein the first layer further comprises the porous material.

6. The endoprosthesis of claim 1, wherein the polymer matrix is formed of a bioerodible polymer.

7. The endoprosthesis of claim 1, wherein the porous material comprises oxides and hydroxides of titanium, iridium, zirconium, ruthenium, hafnium, silicon, and aluminum.

8. The endoprosthesis of claim 1, wherein the surface is formed of a metal.

9. The endoprosthesis of claim 8, wherein the metal is stainless steel, nitinol, tungsten, tantalum, rhenium, iridium, silver, gold, bismuth, platinum or alloys thereof.

10. The endoprosthesis of claim 1, wherein the at least one particle has a diameter of about 10 nm to about 1 μm.

11. The endoprosthesis of claim 1, wherein the plurality of polymeric particles form a layer with a thickness of about 10 nm to about 1 μm.

12. The endoprosthesis of claim 1, wherein the first layer has a thickness of about 1 nm to about 100 nm.

13. The endoprosthesis of claim 1, wherein the porous material has a porosity of about 90% or less.

14. The endoprosthesis of claim 1, wherein the porous material has a porosity of about 30% or more.

15. A method making an endoprosthesis, the method comprising:
applying a first layer of a polyelectrolyte to a surface;
applying a plurality of bioerodible particles to the first layer; and
forming a porous top coating over the particles.

16. The method of claim 15, further comprising applying a second layer of a polyelectrolyte to the particles and applying a precursor composition over the second layer to form the porous top coating via an in situ sol-gel reaction.

17. The method of claim 15, wherein at least one of the bioerodible particles comprises a polymeric matrix and a drug dispersed in the matrix.

18. The method of claim 17, further comprising forming the at least one of the bioerodible particles with the drug dispersed in the matrix by emulsion-polymerization.

19. The method of claim 15 comprising forming the porous top coating by applying a sol-gel precursor composition to form a wet gel.

20. The method of claim 19, further comprising converting the wet gel to a ceramic or ceramic-like material by drying the wet gel.

21. The method of claim 19, wherein the precursor composition comprises a metal oxide precursor.

22. The method of claim 21, wherein the metal oxide precursor is tetraethylorthosilicate, titanium isopropoxide, or iridium acetylacetonate.

23. The method of claim 15 comprising applying the first layer and the particles by LBL deposition.

24. The method of claim 16 comprising applying the second layer by LBL deposition.

25. The method of claim 15, wherein the particle has a size of about 10 nm to about 1 μm.

26. The method of claim 15, wherein the first layer has a thickness of about 1 nm to about 100 nm.

27. The method of claim 15, wherein the porous top coating has a porosity of about 90% or less.

28. The method of claim 15, wherein the porous top coating has a porosity of about 30% or more.
